(19) United States
(12) Patent Application Publication
(10) Pub. No.: US 2010/0069779 A1
(43) Pub. Date: Mar. 18, 2010

(54) Soft tissue moisture analyzer

(76) Inventor: Zvi Nachum, Tiberias (IL)

Correspondence Address: EITAN MEHULAL LAW GROUP, 10 Abba Eban Blvd. PO Box 2081, Herzlia, 46120, IL

(21) Appl. No.: 12/232,280

(22) Filed: Sep. 15, 2008

(51) Int. Cl. A61B 5/053

(52) U.S. Cl. 600/547

(57) ABSTRACT

A device and method for evaluating the moisture of soft tissue within a mouth of a subject, the device including: (a) an alternating current source, adapted to produce an alternating current; (b) an electrode arrangement having at least first and second electrodes, separated by an electrically insulating region, the arrangement having an at least semi-rigid region that fixes the electrodes in a spaced-apart manner, and (c) a processor, wherein the first electrode is electrically connected to the alternating current source, and the second electrode is electrically connected to the processor, and wherein, when the electrode arrangement is provided with the alternating current, and is disposed against the soft tissue, the soft tissue electrically bridges between the electrodes to form an electrical circuit, wherein an electrical signal is produced by: the alternating current passing from the first electrode to the second electrode via the soft tissue, and wherein the processor is adapted to receive electrical information originating from the electrical signal, via the circuit, and to produce an output relating to the moisture of the soft tissue, based on the electrical information.

### FIELD AND BACKGROUND OF THE INVENTION

The present invention relates to an instrument for measuring the moisture of soft tissue in the mouth of a patient.

Sialometers measure the rate of secretion of saliva from the major salivary glands. Typically, sialometers have an arrangement for attaching to the opening of the salivary duct, and a drop counter or other means of measuring the weight or volume of the secretions.

Even using available sialometers, clinicians may not be able to reliably measure the amount of saliva produced by a person (see, for example, Carrie Brown, et al., “The Chin Dry System and Swallow Frequency Device: Technology for Saliva Overflow Problems”, Technology and Persons with Disabilities Conference 1999, California State University, Northridge). Accurate and convenient measurement would enable health care professionals to diagnose various conditions with greater facility, and to better assess the severity of various health problems. Accurate and convenient measurement would further enable health care professionals to better match interventions and treatments to the true state of the patient, and to assess the salivary response of the patient to various drugs or various treatment programs.

### SUMMARY OF THE INVENTION

According to the teachings of the present invention there is provided a device for evaluating a parameter associated with moisture of soft tissue within a mouth of a subject, the device including: (a) an alternating current source, adapted to connect to a power supply and to produce an alternating current; (b) an electrode arrangement having at least a first electrode and a second electrode, the first electrode electrically separated from the second electrode by an electrically insulating region, the arrangement having an at least semi-rigid region that fixes the electrodes in a spaced-apart manner, the arrangement adapted to contact the soft tissue within the mouth of the subject, the electrodes and the insulating region composed of biocompatible materials, and (c) a processor, associated with the electrode arrangement, wherein a first lead from the first electrode is, electrically connected to the alternating current source, and a second lead from the second electrode is electrically connected to the processor, and wherein, when the electrode arrangement is provided with the alternating current, and is disposed against the soft tissue, the soft tissue electrically bridges between the electrodes to form an electrical circuit, wherein an electrical signal is produced by the alternating current passing from the first electrode to the second electrode via the soft tissue, and wherein the processor is adapted to receive electrical information originating from the electrical signal, via the circuit, and to produce an output relating to, or derived from, the parameter, based on the electrical information.

According to another aspect of the present invention there is provided a method for evaluating a parameter associated with moisture of soft tissue within a mouth of a subject, the method including the steps of: (a) providing a device including: (i) an alternating current source, adapted to connect to a power supply and to produce an alternating current; (ii) an electrode arrangement having at least a first electrode and a second electrode, the first electrode electrically separated from the second electrode by an insulating region, the arrangement having an at least semi-rigid region that fixes the electrodes in a spaced-apart manner, the arrangement adapted to contact the soft tissue within the mouth of the subject, (iii) a processor, associated with the electrode arrangement; wherein a first lead from the first electrode is electrically connected to the alternating current source, and a second lead from the, second electrode is electrically connected to the processor; (b) inserting the electrode arrangement into the mouth, against the soft tissue, wherein the soft tissue electrically bridges between the electrodes to form an electrical circuit, (c) passing the alternating current from the first electrode to the second electrode via the soft tissue, producing an electrical signal; (d) receiving, by the processor, electrical information originating from the electrical signal, via the circuit, and (e) computing, by the processor, a representation of the parameter associated with the moisture of soft tissue within the mouth of the subject, based on the electrical information.

According to further features in the described preferred embodiments, the device further includes an analog-to-digital conversion unit, electrically connected to the electrical circuit, and adapted to convert the electrical signal from an analog form to a digital form.

According to still further features in the described preferred embodiments, the parameter associated with soft tissue within the mouth includes an impedance of the current through the soft tissue.

According to yet still further features in the described preferred embodiments, the parameter associated with soft tissue within the mouth includes a level of moisture within the soft tissue.

According to yet still further features in the described preferred embodiments, the processor is designed and configured to compute a representation of a level of moisture in the soft tissue within the mouth of the subject, at least partially based on the electrical information.

According to yet still further features in the described preferred embodiments, the output includes a value based on a relative moisture scale.

According to yet still further features in the described preferred embodiments, the processor correlates a rate of saliva production to the level of moisture within the soft tissue.

According to yet still further features in the described preferred embodiments, the device further includes: a display, electrically associated with the processor, and adapted to display the output.

According to yet still further features in the described preferred embodiments, the device further includes: a display, electrically associated with the processor, and adapted to display the rate of saliva production.

According to yet still further features in the described preferred embodiments, the device further includes a capacitor, electrically disposed between the electrode arrangement and the processor, the capacitor having a capacitance to pass an output signal to the processor, when the electrical signal is above a pre-defined threshold.

According to yet still further features in the described preferred embodiments, the device further includes an adaptor, electrically connected to the alternating current source, the adaptor having an engagement mechanism adapted to physically hold a portion of the arrangement and to electrically connect the electrode arrangement to the current source and to the processor.

According to yet still further features in the described preferred embodiments, the engagement mechanism is adapted to releasably and reversibly engage the arrangement.

According to yet still further features in the described preferred embodiments, an end of the arrangement has an attachment geometry that is complementary to an attachment geometry of the engagement mechanism.

According to yet still further features in the described preferred embodiments, the alternating current source is adapted to deliver the alternating current having a frequency between 100 Hz and 15,000 Hz, more preferably between 300 Hz and 10,000 Hz, and most preferably, between 500 Hz and 5,000 Hz.

According to yet still further features in the described preferred embodiments, the electrodes are disposed on an at least semi-rigid substrate.

According to yet still further features in the described preferred embodiments, the dimensions of a large face of the electrode arrangement, including the substrate, are less than 80 mm by 30 mm.

According to yet still further features in the described preferred embodiments, the thickness of the electrode arrangement, including the substrate, is less than 1.5 mm, preferably less than 1.0 mm, and typically less than 0.8 mm.

According to yet still further features in the described preferred embodiments, the method further includes displaying the representation of the parameter associated with the moisture of soft tissue within the mouth of the subject.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are used to designate like elements.

In the drawings:

FIG. 1 is a schematic block diagram of one aspect of the moisture-analyzing device, according to the present invention;

FIG. 2a is a schematic top view of an electrode arrangement and plug, according to a preferred embodiment of the present invention;

FIG. 2b is a schematic side view of the electrode arrangement and plug provided in FIG. 2a, and

FIG. 3 is a logical flow diagram showing one aspect of the method of the present invention.

### DESCRIPTION OF THE PREFERRED EMBODIMENTS

The principles and operation of the inventive instrument for measuring the moisture of soft tissue in the mouth may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

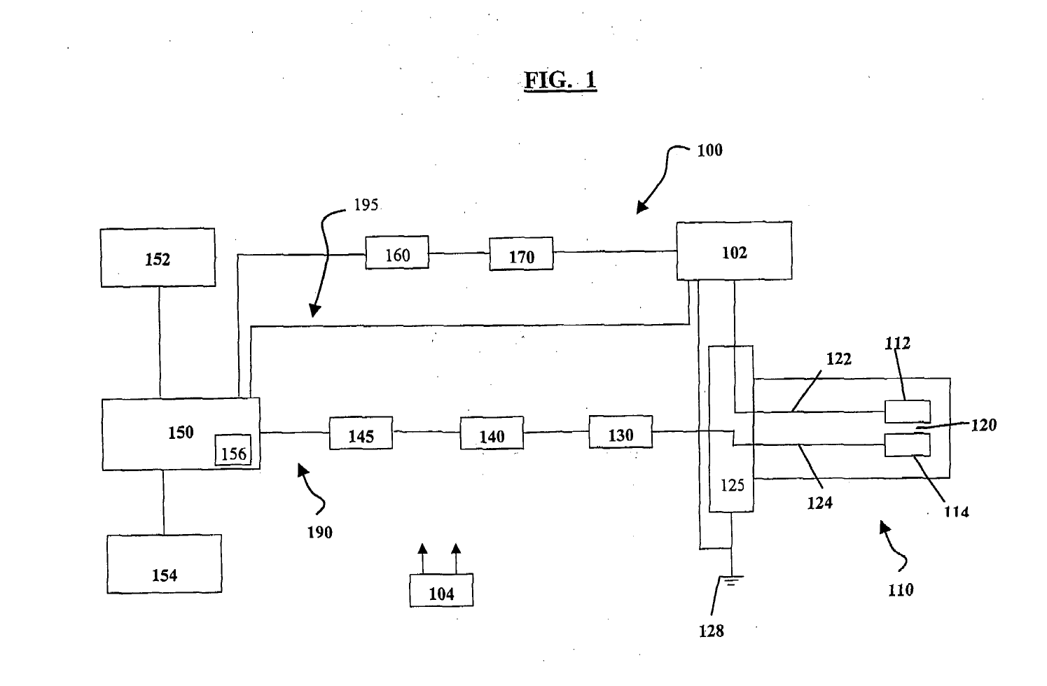

Referring now to the drawings, FIG. 1 is a schematic block diagram of one aspect of a device such as moisture-analyzing device 100, according to the present invention. Moisture-analyzing device 100 includes a current source such as alternating current source 102, connected to a power supply 104 and adapted to produce an alternating current, and an electrode arrangement 110 having at least a first electrode 112 and a second electrode 114 set apart at a fixed distance. First electrode 112 is electrically separated from second electrode 114 by an insulating region 120 having a specific electrical resistivity of at least 1.0 ohm cm, and more typically, at least 10 ohm cm or even 106 ohm cm. Presently preferred materials for insulating region 120 include various biocompatible materials, including polymeric materials such as polypropylene and polycarbonates.

Electrode arrangement 110 has a first lead 122 from first electrode 112 and a second lead 124 from second electrode 114, first lead 122 being electrically connected to alternating current source 102. Electrode arrangement 110 may advantageously be connected to alternating current source 102 by means of an adaptor or plug 125, which will be described in greater detail hereinbelow.

Electrode arrangement 110 is also electrically connected to a processor, such as central processing unit (CPU) 150, which is adapted to receive electrical information originating from the electrical signal, via an electrical circuit 190, and to compute a representation of the level of moisture in the soft tissue within the mouth of the subject, based on the electrical information. To this end, a voltage measuring device, such as voltmeter 156, may advantageously be disposed on circuit 190, or within processor 150, to measure a voltage of the electrical signal or information.

Second lead 124 may be electrically disposed between second electrode 114 and CPU 150. Both current source 102 and second electrode 114 may be connected to a ground 128.

Within, or otherwise electrically associated with CPU 150 may be a memory unit adapted to store data, e.g., data pertaining to electrical parameters, to individual or collective patient parameters or history, etc. Electrically associated with CPU 150 may be a display unit 152 and an input unit 154. Display unit 152, which may be of various types known in the art, including LED and LCD displays, may display an output from CPU 150, such as a calculated impedance between first electrode 112 and second electrode 114, a level of moisture in the soft tissue within the mouth of the subject, or an estimated rate of saliva production.

Input unit 154 may be of various types known in the art, and may be used to select display options, and to provide information to CPU 150. Such information may include data on a particular patient undergoing the test, or the identity of the particular patient.

Electrode arrangement 110 may also be electrically connected to a capacitor 130, which serves to filter currents that are below a pre-determined threshold. A filter such as low pass filter 140 may also be electrically connected to capacitor 130, to filter currents that are above a pre-determined threshold.

The electrical signal from electrode arrangement 110 may be an analog signal, which is converted to a digital signal by means of an analog-to-digital (A2D) converter 145. (A2D) converter 145 may be disposed within CPU 150, or outside CPU 150, as shown. The digital signal is then provided to a processing unit of CPU 150.

When electrode arrangement 110 is inserted into the mouth of a subject, against soft tissue on an inner surface of the mouth, the soft tissue electrically bridges between the electrodes to complete electrical circuit 190. The resulting output signal is provided to CPU 150 via electrical circuit 190.

Preferably, the current source of electrical circuit 190 is an alternating current source such as alternating current source 102. I have found that the soft tissues within the mouth display an electrical behavior having a resistance component and a capacitance component. Direct current is suitable for measuring the resistance component, but may not be suitable for measuring the capacitance component. However, an alternating current source is suitable for measuring both the resistance component and the capacitance component. The frequency of the alternating current source is preferably between 100-15,000 Hz, more preferably, between 300-10,000 Hz, and most preferably, between 500-5,000 Hz.

Moreover, I have surprisingly discovered that when electrode arrangement 110 is provided with the alternating current, and is disposed against soft tissue within the mouth, the output signal is largely unaffected by the particular disposition of the electrodes. In other words, the output signal remains roughly constant, whether the electrodes are disposed on or underneath the tongue, on the gums, or anywhere on the inner surface of the cheek.

I have further surprisingly discovered that this output signal may be strongly correlated to the production rate of saliva produced in the salivary glands. As a first approximation, the correlation is substantially linear, for most subjects.

A secondary, control circuit 195, may be advantageous in controlling the parameters of alternating current source 102 within working limits. Secondary circuit 195 may include CPU 150 and alternating current source 102, along with a low pass filter 160 and a resistor 170 disposed therebetween, to facilitate correction and control of alternating current source 102 by CPU 150.

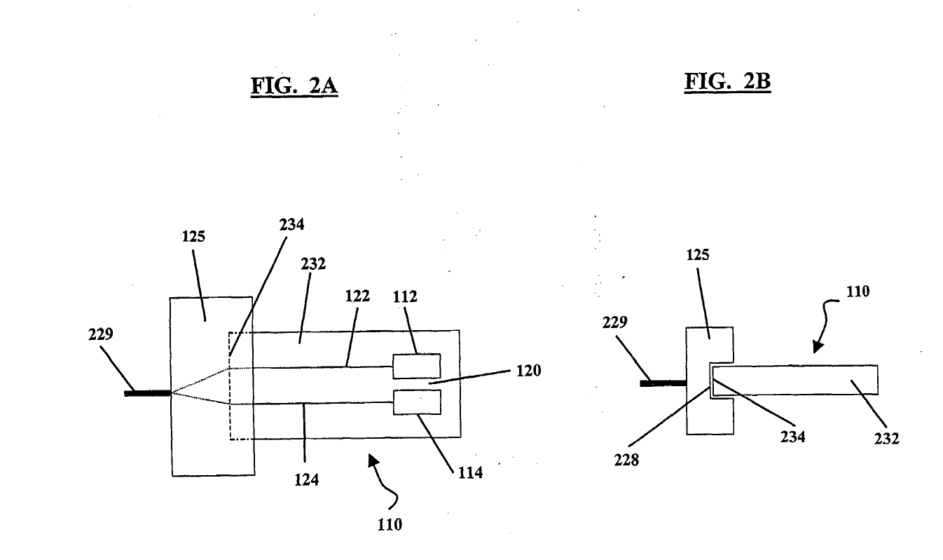

FIG. 2a is a schematic top view of a preferred embodiment of electrode arrangement 110 and plug 125. Electrode arrangement 110 may include a thin, at least semi-rigid substrate 232, typically in the form of a stick or plate, for carrying electrodes 112, 114. It may be advantageous for substrate 232 to exhibit flexibility, at least along a wide face thereof, so as to substantially conform to an inner surface of the mouth. However, insulating region 120 must be sufficiently rigid to maintain the electrodes in a substantially fixed, spaced-apart position.

Presently preferred materials for substrate 232 include various biocompatible materials, including polymeric materials such as polypropylene and polycarbonates.

Electrodes 112, 114 are advantageously made of a highly conducting, biocompatible material such as gold, platinum, copper, silver, as well as various alloys and mixtures containing such materials.

Plug 125 may both mechanically and electrically connect electrode arrangement 110 to alternating current source 102. FIG. 2b provides a schematic side view of electrode arrangement 110 and plug 125 according to an exemplary, preferred embodiment. A first end 234 of substrate 232 engages with an engagement surface 228 of plug 125. In FIG. 2b, first end 234 of substrate 232 is received by engagement surface 228, which may be substantially complementary to at least a portion of first end 234. Engagement surface 228 may exert a pressure on first end 234 of substrate 232 to fix electrode arrangement 110 in place. Other connecting mechanisms for securing substrate 232 to plug 125 will be apparent to those skilled in the art of mechanical connection.

Plug 125 may attach to alternating current source 102 via flexible connecting wire 2-29, which may advantageously be of sufficient length to enable the insertion of electrode arrangement 110 into the mouth of the subject, without having to draw the entire device 100 near to the mouth.

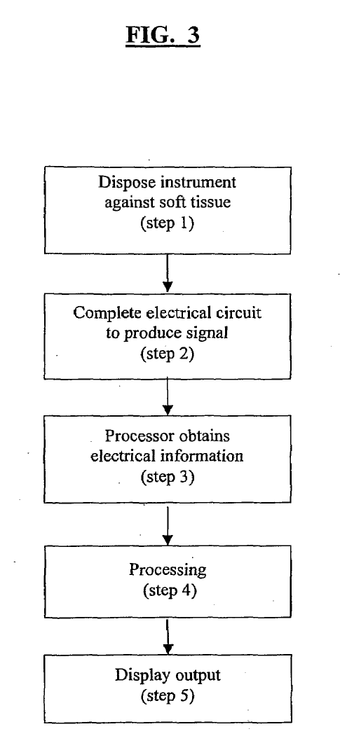

One aspect of the method of the present invention will now be described, with reference to the logical flow diagram provided in FIG. 3 (all numbered device components appear in FIG. 1). Using a device such as moisture-analyzing device 100, an electrode arrangement such as electrode arrangement 110 is inserted into the mouth of the subject and disposed against soft tissue within the mouth, such as on or underneath the tongue, or on the inner surface of the cheek (step 1). When alternating current source 102 is activated, the soft tissue between and generally around the electrodes electrically bridges between the electrodes to complete electrical circuit 190. The alternating current passes from the first electrode to the second electrode via the soft tissue, producing an electrical signal (step 2).

Processor or CPU 150 receives this signal, or electrical information derived from the electrical signal, via circuit 190 (step 3), and then processes the electrical signal, possibly along with other information, to produce an output relating to, or derived from, the parameter associated with soft tissue within the mouth of the subject (step 4). This output may then be displayed (step 5) by display unit 152.

The output may be in the form of a calculated impedance between the electrodes, a level of moisture or moisture rating in the soft tissue, an estimated rate of saliva production, or in other forms. The output may be essential in diagnosing various health conditions of the patient, and in assessing the severity of various health problems. Also, the output may aid in the matching of interventions and treatments to the true state of the patient, as well as in monitoring and determining the salivary response of the patient to drugs and treatment programs of various kinds.

Processor 150 may calculate the electrical impedance (Z) based on the relationship:

Z=R+iX

where R is the ohmic resistance and X is the reactance. In the mouth, the reactance term stems solely from capacitance, hence,

Z=R+iX_{c}

where X_{c} is the capacitance of the circuit between the electrodes.

In any event, when processor 150 is provided with the current (I) provided by alternating current source 102, along with a voltage signal from circuit 190, the impedance may be calculated from the ratio of the two, according to the relationship:

Z=V/I

where V is a voltage associated with the voltage signal. The ratio of voltage to current has been found to strongly correlate with tissue moisture.

I have monitored the moisture of the tissue over the course of the moistening cycle, i.e., the period in between successive swallowings of saliva. Surprisingly, I have discovered that when electrode arrangement 110 is provided with the alternating current, and is disposed against soft tissue within the mouth, the output signal is largely unaffected by the particular time that the electrical measurement is made within the moistening cycle. Consequently, the device and method of the present invention may provide results that are accurate, repeatable, and representative of the state of moisture with the mouth of the subject.

Processor 150 may use the relationship between voltage and current to compute an absolute tissue moisture, or a relative tissue moisture. The relative tissue moisture may be rated, by way of example, on a scale of 1 to 10, or by comparison to the tissue moistures for a particular group, e.g., based on gender, age, etc.

Moreover, in many cases, the relationship between voltage and current, or the calculated tissue moisture, can be correlated to the production rate of saliva, i.e., the rate at which saliva enters the oral cavity. Thus, display unit 152 may display various outputs from CPU 150, including the calculated impedance, a level or rating of moisture in the soft tissue within the mouth, or an estimated rate of saliva production.

As used herein in the specification and in the claims section that follows, the term “electrically connected” refers to a physical connection between elements that enables an electrical current to flow therebetween, when the elements are connected to a current source delivering current.

As used herein in the specification and in the claims section that follows, the term “parameter associated with moisture of soft tissue within the mouth”, and the like, is specifically meant to include a salivary production rate of the subject.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A device for evaluating a parameter associated with moisture of soft tissue within a mouth of a subject, the device comprising:
(a) an alternating current source, adapted to connect to a power supply and to produce an alternating current;
(b) an electrode arrangement having at least a first electrode and a second electrode, said first electrode electrically separated from said second electrode by an electrically insulating region, said arrangement having an at least semi-rigid region that fixes said electrodes in a spaced-apart manner,
said arrangement adapted to contact the soft tissue within the mouth of the subject,
said electrodes and said insulating region composed of biocompatible materials, and
(c) a processor, associated with said electrode arrangement,
wherein a first lead from said first electrode is electrically connected to said alternating current source, and a second lead from said second electrode is electrically connected to said processor,
and wherein, when said electrode arrangement is provided with said alternating current, and is disposed against the soft tissue, the soft tissue electrically bridges between said electrodes to form an electrical circuit, wherein an electrical signal is produced by said alternating current passing from said first electrode to said second electrode via the soft tissue,
and wherein said processor is adapted to receive electrical information originating from said electrical signal, via said circuit, and to produce an output relating to, or derived from, the parameter, based on said electrical information.

2. The device of claim 1, further comprising:
(d) an analog-to-digital conversion unit, electrically connected to said electrical circuit, and adapted to convert said electrical signal from an analog form to a digital form.

3. The device of claim 1, wherein the parameter associated with soft tissue within the mouth includes an impedance of said current through the soft tissue.

4. The device of claim 1, wherein the parameter associated with soft tissue within the mouth includes a level of moisture within the soft tissue.

5. The device of claim 1, wherein said processor is designed and configured to compute a representation of a level of moisture in the soft tissue within the mouth of the subject, at least partially based on said electrical information.

6. The device of claim 5, wherein said output includes a value based on a relative moisture scale.

7. The device of claim 4, wherein said processor correlates a rate of saliva production to said level of moisture-within the soft tissue.

8. The device of claim 1, further comprising:
(d) a display, electrically associated with said processor, and adapted to display said output.

9. The device of claim 7, further comprising:
(d) a display, electrically associated with said processor, and adapted to display said rate of saliva production.

10. The device of claim 1, further comprising:
(d) a capacitor, electrically disposed between said electrode arrangement and said processor, said capacitor having a capacitance to pass an output signal to said processor, when said electrical signal is above a pre-defined threshold.

11. The device of claim 1, further comprising:
(d) an adaptor, electrically connected to said alternating current source, said adaptor having an engagement mechanism adapted to physically hold a portion of said arrangement and to electrically connect said arrangement to said current source and to said processor.

12. The device of claim 11, wherein said engagement mechanism is adapted to releasably and reversibly engage said arrangement.

13. The device of claim 11, wherein an end of said arrangement has an attachment geometry that is complementary to an attachment geometry of said engagement mechanism.

14. The device of claim 1, wherein said alternating current source is adapted to deliver said alternating current having a frequency between 100 Hz and 15,000 Hz.

15. The device of claim 1, wherein said electrodes are disposed on an at least semi-rigid substrate.

16. The device of claim 15, wherein dimensions of a large face of said electrode arrangement, including said substrate, are less than 80 mm by 30 mm.

17. The device of claim 16, wherein a thickness of said electrode arrangement, including said substrate, is less than 1.5 mm.

18. A method for evaluating a parameter associated with moisture of soft tissue within a mouth of a subject, the method comprising the steps of:
(a) providing a device including:
(i) an alternating current source, adapted to connect to a power supply and to produce an alternating current;
(ii) an electrode arrangement having at least a first electrode and a second electrode, said first electrode electrically separated from said second electrode by an insulating region, said arrangement having an at least semi-rigid region that fixes said electrodes in a spaced-apart manner, said arrangement adapted to contact the soft tissue within the mouth of the subject,
(iii) a processor, associated with said electrode arrangement;
wherein a first lead from said first electrode is electrically connected to said alternating current source, and a second lead from said second electrode is electrically connected to said processor;
(b) inserting said electrode arrangement into the mouth, against the soft tissue, wherein the soft tissue electrically bridges between said electrodes to form an electrical circuit,
(c) passing said alternating current from said first electrode to said second electrode via the soft tissue, producing an electrical signal;
(d) receiving, by said processor, electrical information originating from said electrical signal, via said circuit, and
(e) computing, by said processor, a representation of the parameter associated with the moisture of soft tissue within the mouth of the subject, based on said electrical information.

19. The method of claim 18, said alternating current having a frequency between 500 and 5,000 Hz.

20. The method of claim 18, further comprising the step of:
(f) displaying said representation.
